(19) United States
(12) Patent Application Publication
(10) Pub. No.: US 2024/0016441 A1
(43) Pub. Date: Jan. 18, 2024

(54) SODIUM L-LACTATE INFUSION FOR EARLY DIAGNOSIS OF PARKINSON'S DISEASE

(71) Applicant: Joel Steven Goldberg, Hillsborough, NC (US)

(72) Inventor: Joel Steven Goldberg, Hillsborough, NC (US)

(21) Appl. No.: 18/373,387

(22) Filed: Sep. 27, 2023

(51) Int. Cl. A61B 5/00

(52) U.S. Cl. CPC A61B 5/4082; A61B 5/4848

(57) ABSTRACT

Parkinson's disease is characterized by degeneration of highly metabolic dopaminergic neurons which produce clinical signs to include tremor, rigidity, and glabellar reflex. There are few objective, inexpensive, sensitive, and specific tests for early diagnosis of Parkinson's disease. In this invention, patients who manifest early objective signs of possible Parkinson's disease are pretreated with a supplement to increase central nervous system NAD⁺, and then subsequently administered a sodium L-lactate infusion after which the signs of early Parkinson's disease are recorded. Remission of the early signs of Parkinson's disease is diagnostic for Parkinson's disease. A clinical trial is suggested.

### CROSS-REFERENCES TO RELATED APPLICATIONS

This application claims the benefit of U.S. application Ser. Nos. 14/663,911, 16/876,647, and 17/698,053 which are incorporated by reference.

### FEDERALLY FUNDED RESEARCH

Not applicable

### BACKGROUND OF THE INVENTION

### Definitions

The term “lactate” is ambiguous. In this invention “lactate” will be designated as “L-lactate” or “DL-lactate” or “racemic lactate”.

In this invention “L-lactate infusion” is synonymous with “sodium L-lactate infusion” unless otherwise stated.

### Overview of Parkinson's Disease

Parkinson's disease is predicted to affect more than 10% of the population over the age of 80 years. Some causes of Parkinson's disease include genetic predisposition and exposure to toxins, but in the vast majority of patients the cause is not well defined.

Although new and expensive imaging studies to diagnose Parkinson's disease are on the horizon, the main criteria for diagnosis are signs and symptoms. Signs include but are not limited to resting tremor, cogwheel rigidity, bradykinesia, glabellar reflex, postural flexion, and blank facies. Symptoms include but are not limited to depression, dementia, dysphagia, and chronic pain including arthralgia, myalgia, and thermal paresthesia. The instant invention is a method to diagnose early Parkinson's disease based on remission of early objective signs when the energy requirements of dopaminergic neurons are fulfilled with an L-lactate infusion in nicotinamide pretreated patients.

### Energy Requirements of Dopaminergic Neurons

One prevailing concept that may explain the etiology of Parkinson's disease is the precarious energy requirements of dopaminergic neurons. Dopaminergic neurons, as compared to other neurons, are composed of “massive, unmyelinated axonal arbor that is orders of magnitude larger than other neural types”. [1, 2] In addition, dopaminergic neurons have inherent pacemaker activity that consume large amounts of energy. To maintain this structure and function, the energy requirements for dopaminergic neurons are predicted to exceed other neuron classes. Prior art discusses potential treatment of Parkinson's disease with strategies to improve the energy imbalance but does not discuss L-lactate infusion.[2] Although completely different diseases, this predicted neural energy deficit is also proposed to occur in the motor neurons of patients afflicted with amyotrophic lateral sclerosis (ALS) where L-lactate supplementation may improve symptoms.[3-5]

### L-Lactate and Parkinson's Disease

Yamamoto et al. studied L-lactate cerebrospinal fluid (CSF) concentrations in treated or untreated Parkinson's patients and controls, and there were no significant differences.[6] Yang et al. reported elevated L-lactate CSF levels in late-onset Parkinson patients, and Li et al. demonstrated the apoptosis of dopaminergic neurons treated with L-lactate which teaches away from the concepts in the instant invention.[7, 8] A group of serum metabolic biomarkers have been proposed for the early diagnosis of Parkinson's disease that include L-lactate, but this study did not include L-lactate infusion, and serum L-lactate was not a main metabolic discriminating difference between patients with Parkinson's disease and controls.[9] Present medication therapies to ameliorate the signs and symptoms of Parkinson's disease include the administration of levodopa, carbidopa, dopamine agonists, catechol-o-methyltransferase inhibitors, and monoamine oxidase B inhibitors; however, these do not target the suspected energy deficit of dopaminergic neurons and do not include L-lactate.

ATP within the Cytoplasm Vs. ATP within the Mitochondria

It has been previously proposed, but not widely accepted, that aerobic glycolysis is the primary source of adenosine triphosphate (ATP) in cells that are hypermetabolic, and dopaminergic neurons would qualify for this condition considering their metabolic demands.[10, 11] The support for this concept is based upon the physiology that ATP produced from aerobic glycolysis in the cytoplasm is of different quality than ATP produced in the mitochondrial matrix. The rate of production of the latter ATP is regulated by transit of pyruvate from the cytoplasm to the mitochondria and subsequent rates of citric acid cycle oxidation and oxidative phosphorylation, as compared to ATP synthesized in the cytoplasm.

The efflux of the mitochondrial ATP is dependent upon the rate of transport across two mitochondrial membranes to service the energy requirements of organelles within the cytoplasm. The proof of this concept has not been established. An alternative view of cell energetics has been proposed that L-lactate synthesis is the 11^{th} step in glycolysis regardless of oxygen availability in the cytoplasm, and the L-lactate is oxidized in the outer mitochondrial membrane, presumably by mitochondrial LDH to pyruvate, which then proceeds to enter the citric acid cycle.[12] Isotope studies using [3-¹³C] L-lactate with measurement of ¹³CO₂ support brain L-lactate oxidation in normal healthy controls and traumatic brain injury patients.[13]

Thermodynamic vs. Kinetic Products of L-Lactate+NAD⁺↔Pyruvate+NADH

Neurons, and its constituents to include cytoplasm, mitochondria, and other organelles, are open thermodynamic systems where energy and matter are in constant exchange, and applying classical equilibrium thermodynamics to open systems is fraught with many misinterpretations and misconceptions.

L-lactate is well known to cross the blood brain barrier and cell membranes via monocarboxylate transporters in proportion to its concentration gradient.[14] It can also cross cell membranes by free diffusion or ion exchange.[15] L-Lactate has been proposed as an energy source for neurons, especially L-lactate that has been shuttled from astrocytes to neurons. The astrocyte-to-neuron shuttle hypotheses remains controversial in light of thermodynamic calculations for the oxidation of L-lactate to pyruvate.[16, 17] For the L-lactate to be a source of ATP, it needs to be oxidized to pyruvate, a thermodynamically unfavorable reaction that consumes NAD⁺ that is essential for continuation of glycolysis. Even if the oxidation takes place within the mitochondria as opposed to the cytoplasm, there is a net loss of NAD⁺ in the cell. Other mechanisms such as the presence of L-lactate oxidase or NAD⁺ independent LDH, which conserve NAD⁺, may exist.

The standard free energy (ΔG°) of L-lactate oxidation to pyruvate coupled with the reduction of NAD⁺ to NADH is +25.1 kJ/mol reaction:

ΔG=ΔG°+2.303 RT log Q

For the reaction to be favored assuming classical equilibrium thermodynamics:

ΔG<0

ΔG°=25,100 J, R=8.314 J*mol⁻¹ K⁻¹, T=310° K

When ΔG<0

Q<5.9*10⁻⁵

Therefore [pyruvate]/[L-lactate]*[NADH]/[NAD⁺]<5.9*10⁻⁵

Cytoplasmic [NADH]/[NAD⁺] range from 0.0014 to 0.016.[18] The CSF [pyruvate]/[L-lactate] range from 0.038 to 0.110.[19] Using average values [pyruvate]/[L-lactate]*[NADH]/[NAD⁺] would be 6.40*10⁻⁴, clearly larger than 5.9*10⁻⁵.

When [pyruvate][L-lactate] is 7.6*10⁻³ and [NADH]/[NAD⁺] is 7.6*10⁻³ then ΔG<0. The concentration gradients of [pyruvate][L-lactate] and [NADH]/[NAD⁺] required to make the reaction thermodynamically favorable may not be in the range of physiologic concentrations.

LDH1, which is found in neurons, preferentially converts L-lactate to pyruvate, a stereospecific, reversible reaction which is not thermodynamically favored. However, all experiment evidence clearly demonstrates this reaction proceeds in vitro and in vivo under physiologic conditions.[20, 21] The products of the reaction, pyruvate and NADH, can be explained:
1. Biological systems often increase reactants or decrease products to change mass action ratios and overcome classical equilibrium thermodynamic products.
2. A low activation energy associated with a transition state could favor a kinetic product.
3. Asymmetric synthesis products are observed from the forward pyruvate↔L-lactate reaction since D-lactate is not a product, and this favors a kinetic mechanism which may occur in the reverse reaction.
   L-Lactate as a Buffer for H⁺ from Glyceraldehyde-3-Phosphate Dehydrogenase Reaction of Glycolysis

Glycolysis generates free H⁺ from inorganic phosphate (H₂PO₄) when NAD⁺ is reduced in the glyceraldehyde-3-phosphate to 1,3 bisphosphoglycerate reaction.[22] In order for glycolysis to proceed and the cell remain viable, these H⁺ need to be buffered, and the cell needs to maintain its membrane potential. Some of the H⁺ may eventually be transported to the mitochondria and be utilized in the ATP synthase process, but this may not account for the large acid burden. In such instances it seems quite plausible that extracellular L-lactate from astrocytes could be transported into the cytoplasm to maintain pH and membrane potential. This mechanism would not exclude oxidation of L-lactate as a primary energy since both mechanisms could coexist.

### Prior Art of Sodium DL-Lactate and L-Lactate Infusions

There is an abundance of literature describing the medical effects of sodium DL-lactate infusion to include compounded sodium DL-lactate infusions and infusion of Ringer's lactate.[23] These studies are distinct from the instant invention since the sodium DL-lactate is often a racemic mixture and not a sodium L-lactate infusion. Many of the pharmacodynamics and pharmacokinetic properties of L-lactate differ from D-lactate, and thus the results of these studies are not applicable of prior novelty. Some of the literature describing sodium DL-lactate infusions may be flawed since DL-lactate is infused but subsequently L-lactate plasma levels are measured.

Kuze et al. compared infusions of sodium L-lactate and sodium racemic lactate each with total L-lactate or DL-lactate concentrations of 84 mEq/L at 5 ml*kg⁻¹*h⁻¹ for 3.5 hours, achieving a maximum L-lactate whole-blood concentration of 41.3 mg/dL in a single anesthetized patient without any significant change in vital signs. [24]

Dager et al. infused sodium DL-lactate in patients to investigate panic attacks, reporting a peak blood L-lactate concentration of 10.8 mmol/L or 97.2 mg/dL.[23]

Sodium DL-lactate, but not L-lactate, has been infused in male athletes with an increase in BDNF blood concentration p<0.05.[25]

Glenn et al. infused sodium [3-¹³C] L-lactate in patients with traumatic brain injury and healthy controls, attaining a peak L-lactate blood concentration of 10.6 mg/dL without serious complications.[13]

Boumezbeur et al. utilized infusions of sodium [3-¹³C] L-lactate at a concentration of 350 mmol/L with a priming dose of 150 μmol/kg over 5 minutes, followed with a continuous infusion of 10 μmol*kg⁻¹*min⁻¹ for ^{˜}120 min or a priming dose of 300 μmol/kg over 5 minutes followed with a continuous infusion of 20 μmol*kg⁻¹*min⁻¹ for ^{˜}120 minutes, achieving a maximum L-lactate plasma concentration of 24.9 mg/dL without any adverse effects.[26] They concluded plasma to brain concentration of L-lactate was positively linearly correlated, and plasma L-lactate to brain metabolism is 10% under basal plasma L-lactate concentrations of ^{˜}1.0 mmol/L and as much as 60% at high physiologic plasma concentrations. These measurement were obtained in the occipital-parietal lobe where a low concentration of dopaminergic neurons exist, and the results are not applicable to L-lactate infusion effects in patients with Parkinson's disease. Approximately 80% of dopamine in the brain is found in the substantia nigra and the projections to the basal ganglia.

### Prior Art of L-lactate Stress Testing in Parkinson's Disease

L-lactate stress testing patients are subjected to exercise tasks with associated elevation of plasma L-lactate. Blood L-lactate with and without exercise in Parkinson's disease are included in NIH ClinicalTrial.gov (NCT021844944), and L-lactate stress testing effects on neurotrophin BDNF levels have been reported.[27] BDNF serum levels have been used as a potential diagnostic agent in many neural cell injury diseases including Parkinson's disease, but it is not obvious nor predictable that L-lactate infusions would diminish Parkinsonian motor responses by increasing BDNF.[28] The stress studies are not applicable to the novelty of this invention since they do not teach L-lactate infusion, and the plasma levels of L-lactate in stress testing are much lower than by direct infusion 18.6 mg/dL vs. 24.9 mg/dL-41.3 mg/dL. Peak plasma L-lactate levels in L-lactate stress testing may not translate to CSF L-lactate levels since the L-lactate is simultaneously converted to glucose or oxidized to bicarbonate in the liver. It is only through L-lactate infusion that a steady state can be reached in the CSF. [29] Furthermore, these studies did not measure motor responses to the L-lactate infusion.

### Safety of L-Lactate Infusion

The safety of L-lactate infusion has been demonstrated at 24.9 mg/dL in non-anesthetized patients and 41.3 mg/dL in anesthetized patients compared to baseline of 8.1 mg/dL without any adverse effects. The safety of L-lactate infusions as a component of DL-lactate or racemic lactate infusions is also well established.

### Pharmacodynamics of L-Lactate in the Brain

L-Lactate is very likely a significant source of energy in the brain especially when glucose supplies are limited. However, LDH conversion of L-lactate to pyruvate will consume NAD⁺ which is crucial for the continuation of glycolysis. L-Lactate infusions may increase the intraneural levels of L-Lactate that can serve as a buffer for H⁺, thus maintaining membrane potentials and promoting cell integrity especially crucial in hypermetabolic dopaminergic neurons. As previously stated, L-lactate infusions have been performed in human subjects for decades without serious side effects, but not for the diagnosis of Parkinson disease. This invention discloses that L-lactate infusion is a method to diagnose Parkinson's disease where such an infusion will ameliorate three motor signs in a subject suspected of Parkinson's disease who has abundant NAD⁺ stores from pretreatment with nicotinamide.

Effects of L-Lactate not Directly Concerned with Energy Metabolism

L-lactate has been reported to regulate many metabolic functions that are not directly related to ATP production. These include but are not limited to L-lactate as: an immune modulator, an epigenetic modulator via lactylation, a regulator of fatty acid metabolism, a redox buffer, and a G protein receptor agonist. [30] When a substance is reported to possess a myriad of biologic actions, it is important to separate out the essential clinically relevant action from other less important actions.

### D-Lactate and DL-Lactate Infusions

The vast majority of “lactate” infusions in the medical literature are infusions of sodium DL-lactate, most of which contain equal molar amounts of D-lactate and L-lactate. The pharmacologic properties of D-lactate are distinct from L-lactate. The LDH-D enzyme is stereospecific for non-reversible conversion of D-lactate to pyruvate. Elevation of plasma D-lactate is associated with neurotoxicity; however, D-lactate has been reported to increase survival of dopaminergic neurons related to a glyoxalase pathway.[31] Infusion of sodium DL-lactate, sodium racemic lactate, or sodium D-lactate is distinct from the instant invention of L-lactate infusion.

### Advanced Parkinson's Disease and Motor Nervous System

Although the motor nervous system may not be the most studied system in neuroscience, it is argumentatively the most important. The motor system is uniquely the system of the central nervous system (CNS) that interacts with the environment, whether it be through speech, writing, or other movements. The CNS processes information in various regions, but the output is always through the motor system which determines behavior. In very advanced stages of Parkinson's disease, ALS, and Alzheimer's disease, when motor activity is compromised, behavior is severely impaired.

### BRIEF SUMMARY OF THE INVENTION

This invention is a novel, inexpensive, objective method to diagnose early Parkinson's disease, utilizing an intravenous L-lactate infusion and demonstrating remission of motor responses associated with early Parkinson's disease. L-lactate infusion is predicted to preserve the high energy balance in dopaminergic neurons either as a direct fuel source or through buffering of intracellular H⁺, and upregulate aerobic glycolysis. Early diagnosis of Parkinson disease may improve overall management of this progressive disease. Furthermore, proof of the energy hypothesis as the etiology of Parkinson's disease could open the door to the development of new pharmacophores that may supplement present therapies and improve patient outcome. A clinical trial is suggested.

### BRIEF DESCRIPTION OF THE SEVERAL VIEWS OF THE DRAWING

Not applicable

### DETAILED DESCRIPTION OF THE INVENTION

The concepts of the instant invention can be summarized as follows:
1. The etiologies of Parkinson's disease are many; however, the underlying dysfunction of dopaminergic neurons can be explained by the hypermetabolic energy imbalance requirements of these dopaminergic neurons, which are structurally and functionally distinct from other neurons.
2. L-lactate can restore the energy deficit in dopaminergic neurons, either directly as a fuel to increase ATP, or as a buffer to increase the efficiency of aerobic glycolysis.
3. Restoring the energy balance in compromised dopaminergic neurons can be clinically assessed by measuring motor functions including, resting tremor, cogwheel rigidity, and glabellar reflex.

The early diagnosis of Parkinson's disease is not easy, requiring careful examination skills even challenging a very skilled neurologist. One criteria includes bradykinesia and either tremor or rigidity. There are no good laboratory tests, and brain scans including advance dopamine imaging is expensive and not conclusive. A challenge with levodopa therapy is sometimes required to confirm the diagnosis of Parkinson's disease, and such a challenge is not without substantial risks.[32] In the instant invention, three objective physical examination findings are measured to include resting tremor, cogwheel rigidity and/or glabellar reflex, and remission of the motor responses after L-lactate infusion is diagnostic of Parkinson's disease.

Three common forms of tremors observed in patients with Parkinson's disease include resting tremors, postural tremors, and action tremors. Resting tremors with combined frequencies of 4-5 Hz and 6 Hz diagnose Parkinson's disease in greater than 80% of patients with tremor; however, tremor frequency is not easy to assess in most clinical situations, which confirms the utility of the instant invention. Resting tremor needs to be separated from tremor of other causes.[33]

Cogwheel rigidity is a hallmark of Parkinson's disease. It can be assessed by flexion of the wrist or elbow and commonly occurs in the frequencies of 6 Hz and 8-9 Hz, which are not easily assessed in the clinic. It needs to be distinguished between rigidity from pseudo-parkinsonism which is one utility of the instant invention.

Although the glabellar reflex has been used as a diagnostic criteria by neurologists for decades, its use is controversial with poor correlation with (DAT) [¹²³I] FP-CIT SPECT scanning. It poorly discriminates between patients with essential tremor vs. Parkinsonian tremor.[34]However, clinical studies demonstrate reversal of the glabellar reflex with successful levodopa treatment in patients with Parkinson's disease.[35] This reflex is easily quantified and can be easily conducted at the bedside. Incorporation of this reflex in the instant invention teaches away from present diagnostic testing for Parkinson's disease.

### Steps in the Method to Diagnose Early Parkinson's Disease

1. Three signs of suspected Parkinson's disease are evaluated on numeric scales and recorded. These signs include resting tremor, cogwheel rigidity, and glabellar reflex.
2. Patients are subsequently treated to increase CSF NAD⁺ with nicotinamide, the preferred embodiment, or other agents known to increase NAD⁺ levels to include but not limited to nicotinic acid, nicotinamide riboside, or nicotinic acid riboside.[18]
3. The three signs of suspected Parkinson's disease are reevaluated and recorded.
4. An isotonic, intravenous solution comprised of L-lactate infusion is administered with an initial loading dose 300 μmol/kg over 5 minutes followed with a continuous infusion of 20 μmol*kg⁻¹*min⁻¹ for ^{˜}120 minutes.
5. After L-lactate infusion, signs of resting tremor, cogwheel rigidity, and glabellar reflex are evaluated and recorded.
6. Remission of resting tremor, cogwheel rigidity, and/or glabellar reflex is diagnostic of Parkinson's disease.

### REFERENCES

1. Pissadaki, E. K. and J. P. Bolam, The energy cost of action potential propagation in dopamine neurons: clues to susceptibility in Parkinson's disease. Front Comput Neurosci, 2013. 7: p. 13.
2. Mamelak, M., Parkinson's Disease, the Dopaminergic Neuron and Gammahydroxybutyrate. Neurol Ther, 2018. 7(1): p. 5-11.
3. Goldberg, J. S., TREATMENT OF AMYOTROPHIC LATERAL SCLEROSIS WITH LACTATE, application Ser. No. 14/663,911, US 2016/0271085 A1.
4. Tefera, T. W. and K. Borges, Metabolic Dysfunctions in Amyotrophic Lateral Sclerosis Pathogenesis and Potential Metabolic Treatments. Front Neurosci, 2016. 10: p. 611.
5. Vadakkadath Meethal, S. and C. S. Atwood, Lactate dyscrasia: a novel explanation for amyotrophic lateral sclerosis. Neurobiol Aging, 2012. 33(3): p. 569-81.
6. Yamamoto, M., et al., Cerebrospinal fluid lactate and pyruvate concentrations in patients with Parkinson's disease and mitochondrial encephalomyopathy, lactic acidosis, and stroke-like episodes (MELAS). J Neurol Neurosurg Psychiatry, 1997. 62(3): p. 290.
7. Yang, C., et al., Lactate metabolism in neurodegenerative diseases. Neural Regen Res, 2024. 19(1): p. 69-74.
8. Li, J., et al., Upregulated hexokinase 2 expression induces the apoptosis of dopaminergic neurons by promoting lactate production in Parkinson's disease. Neurobiol Dis, 2022. 163: p. 105605.
9. Mallet, D., et al., A metabolic biomarker predicts Parkinson's disease at the early stages in patients and animal models. J Clin Invest, 2022. 132(4).
10. Goldberg, J. S., INHIBITION OF GLYCOLYSIS WITH 2-DEOXY-D-GLUCOSE AND D-LACTIC ACID DIMER, application Ser. No. 16/876,647, US 2020/0276216 A1.
11. Goldberg, J. S., AEROBIC GLYCOLYSIS AND HYPERMETABOLIC STATES, application Ser. No. 17/698,053, US 2022/0206017 A1.
12. Schurr, A., From rags to riches: Lactate ascension as a pivotal metabolite in neuroenergetics. Front Neurosci, 2023. 17: p. 1145358.
13. Glenn, T. C., et al., Lactate: brain fuel in human traumatic brain injury: a comparison with normal healthy control subjects. J Neurotrauma, 2015. 32(11): p. 820-32.
14. Kane, D. A., et al., Lactate shuttling as an allostatic means of thermoregulation in the brain. Front Neurosci, 2023. 17: p. 1144639.
15. Manosalva, C., et al., Role of Lactate in Inflammatory Processes: Friend or Foe. Front Immunol, 2021. 12: p. 808799.
16. Bak, L. K. and A. Schousboe, Misconceptions regarding basic thermodynamics and enzyme kinetics have led to erroneous conclusions regarding the metabolic importance of lactate dehydrogenase isoenzyme expression. J Neurosci Res, 2017. 95(11): p. 2098-2102.
17. Bak, L. K. and A. B. Walls, CrossTalk opposing view: lack of evidence supporting an astrocyte-to-neuron lactate shuttle coupling neuronal activity to glucose utilisation in the brain. J Physiol, 2018. 596(3): p. 351-353.
18. Yang, Y. and A. A. Sauve, NAD(+) metabolism: Bioenergetics, signaling and manipulation for therapy. Biochim Biophys Acta, 2016. 1864(12): p. 1787-1800.
19. Zhang, W. M. and M. R. Natowicz, Cerebrospinal fluid lactate and pyruvate concentrations and their ratio. Clin Biochem, 2013. 46(7-8): p. 694-7.
20. Howell, B. F., S. McCune, and R. Schaffer, Lactate-to-pyruvate or pyruvate-to-lactate assay for lactate dehydrogenase: a re-examination. Clin Chem, 1979. 25(2): p. 269-72.
21. Forkasiewicz, A., et al., The usefulness of lactate dehydrogenase measurements in current oncological practice. Cell Mol Biol Lett, 2020. 25: p. 35.
22. Lane, A. N., T. W. M. Fan, and R. M. Higashi, Metabolic acidosis and the importance of balanced equations. Metabolomics, 2009. 5(2): p. 163-165.
23. van Gemert, L. A., et al., Lactate infusion as therapeutical intervention: a scoping review. Eur J Pediatr, 2022. 181(6): p. 2227-2235.
24. Kuze, S., et al., Effects of sodium L-lactate and sodium racemic lactate on intraoperative acid-base status. Anesth Analg, 1992. 75(5): p. 702-7.
25. Schiffer, T., et al., Lactate infusion at rest increases BDNF blood concentration in humans. Neurosci Lett, 2011. 488(3): p. 234-7.
26. Boumezbeur, F., et al., The contribution of blood lactate to brain energy metabolism in humans measured by dynamic 13C nuclear magnetic resonance spectroscopy. J Neurosci, 2010. 30(42): p. 13983-91.
27. Muller, P., et al., Lactate and BDNF: Key Mediators of Exercise Induced Neuroplasticity? J Clin Med, 2020. 9(4).
28. Jiang, L., et al., Serum level of brain-derived neurotrophic factor in Parkinson's disease: a meta-analysis. Prog Neuropsychopharmacol Biol Psychiatry, 2019. 88: p. 168-174.
29. Finsterer, J., et al., Lactate stress test in the diagnosis of mitochondrial myopathy. J Neurol Sci, 1998. 159(2): p. 176-80.
30. Li, X., et al., Lactate metabolism in human health and disease. Signal Transduct Target Ther, 2022. 7(1): p. 305.
31. Toyoda, Y., et al., Products of the Parkinson's disease-related glyoxalase DJ-1, D-lactate and glycolate, support mitochondrial membrane potential and neuronal survival. Biol Open, 2014. 3(8): p. 777-84.
32. Su, D., et al., Age as a risk factor for orthostatic hypotension induced by the levodopa challenge test in patients with Parkinson's disease: Results from a single-center trial. Medicine (Baltimore), 2023. 102(9): p. e33161.
33. Findley, L. J., M. A. Gresty, and G. M. Halmagyi, Tremor, the cogwheelphenomenon and clonus in Parkinson's disease. J Neurol Neurosurg Psychiatry, 1981. 44(6): p. 534-46.
34. Nuuttila, S., et al., Diagnostic accuracy of glabellar tap sign for Parkinson's disease. J Neural Transm (Vienna), 2021. 128(11): p. 1655-1661.
35. Klawans, H. L., Jr. and J. A. Goodwin, Reversal of the glabellar reflex in Parkinsonism by L-dopa. J Neurol Neurosurg Psychiatry, 1969. 32(5): p. 423-7.

Having described my invention, I claim:

1. A method to diagnose Parkinson's disease in a human subject comprising:
administering nicotinamide or an equivalent, derivative or analog thereof to the subject;
infusing sodium L-lactate or a salt of L-lactate via an intravenous access to the subject; and
recording a decrement in a Parkinson motor response in the subject.

2. The method of claim 1, wherein an equivalent, derivative or analog thereof is nicotinic acid, nicotinamide riboside, or nicotinic acid riboside.

3. The method of claim 1, wherein a Parkinson motor response is tremor, cogwheel rigidity, or glabellar reflex.

4. The method of claim 1, wherein an equivalent, derivative or analog thereof is nicotinic acid, nicotinamide riboside, or nicotinic acid riboside and wherein a Parkinson motor response is tremor, cogwheel rigidity, or glabellar reflex.
